(19) United States
(12) Patent Application Publication
(10) Pub. No.: US 2002/0197741 A1
(43) Pub. Date: Dec. 26, 2002

(54) Method of determining time of death

(76) Inventors: Albert J. Sabucedo, Miami, FL (US); Kenneth G. Furton, Miami, FL (US)

Correspondence Address: Mark H. Hopkins, MARSHALL, GERSTEIN & BORUN, Sears Tower, Suite 6300, 233 S. Wacker Drive, Chicago, IL, 60606-6357, US

(21) Appl. No.: 10/144,346

(22) Filed: May 10, 2002

Related U.S. Application Data
(60) Provisional application No. 60/290,370, filed on May 11, 2001.

(51) Int. Cl. G01N 33/53; G01N 33/567; G01N 33/553

(52) U.S. Cl. 436/526

(57) ABSTRACT

A method of determining time of death is disclosed. Cardiac troponin I (cTnI) is the most specific marker for the determination of myocardial infarction (MI). Cardiac troponin I has a distinctive temporal degradation profile after death, which is key to its use as a time of death marker in forensic medicine. The method consists of sampling cardiac tissue, extracting a cardiac protein from a homogenate via methods which may include the magnetic microparticles or magnetic microparticles linked to anti-cTnI antibodies, eluting the proteins, separating proteins by electrophoresis, transferring by western blot the different bands of proteins to paper. A comparison/analysis of the concentration and kinetics of degradation of the protein at a given temperature(s) against known standards after time of death provides an accurate measurement of the time of death. The present invention is more accurate and reliable than the rudimentary time of death techniques currently used by medical examiners.

### CROSS-REFERENCE TO RELATED APPLICATION

This application claims the benefit of provisional patent application Serial No. 60/290,370, filed May 11, 2001.

### FIELD OF THE INVENTION

The present invention relates generally to methods and measuring instruments for use in forensic medical applications, and particularly to methods, instruments, and the use of instruments to determine the time of death.

### BACKGROUND OF THE INVENTION AND PRIOR ART

Globally approximately 55 million people die per year. In the United States, there were 6.9 million deaths between 1993 and 1995. The number of deaths that resulted from homicides, suicides, and unintentional injuries in this same period was about 432,000 or 6.3% of the total number of deaths. Unintentional injuries and suicides ranked among the top 10 leading causes of death in the U.S. and the U.S. homicide rate remained as one of the highest in all of the industrialized countries. An accurate and reliable estimation of the time since death (postmortem interval, PMI) is of great importance in both criminal and civil cases in the U.S. and elsewhere in the world.

An accurate and reliable estimation of the time since death is one of the most challenging problems facing forensic pathology today. When compared with other medical fields, the field of time of death markers has lagged behind the great advances in medical technology that has occurred since the late 1850's (Knight B. “The Estimation of the Time of Death in the Early Postmortem Period” Oxford University Press, Inc., New York 1994, p.1). A “time of death marker” can be a physical, chemical or biochemical parameter that changes upon death and can be measured to determine the time of death with some certainty. Today, medical examiners still use rudimentary time of death marker techniques that offer little, if any, reliability in the medico-legal arena. The most commonly used methods are temperature drop based models which describe the cooling of the body after death. Specifically, the drop in the body temperature of a cadaver is calculated and then divided by 1.5 to give an approximate value of the number of hours since death. This method is notorious for its inaccuracy and can never be more than an approximation of the time of death (Marshall T K. “Estimating the time of death” Journal of Forensic Science 1962;7:189-210). Some of the issues with temperature based methods are the many variables that affect the rate of the drop in temperature, which include the amount and type of clothing worn, the environment that the body is in, the amount of body fat, whether the deceased had a fever at the time of death, etc.

Several more recent reports have focused on postmortem biochemical changes as markers to establish the time of death, however, these techniques suffer from a great number of issues that make them less than ideal candidates as time of death markers. Gallois-Montbrun et al. (“Postmortem interval estimation by biochemical determination in birds muscle” Forensic Science International 1988; 37:189-192) explored several biochemical markers in hen's pectoral muscle. Their results showed that protein, urea, bilirubin, glucose, iron, potassium, calcium alanine amino transferase (ALAT), lactic dehydrogenase (LDH), creatinine phosphokinase (CPK) and alkaline phosphatase (ALP) did not have a significant correlation with PMI, but a strong correlation was found with creatine. Attempts at using electrolyte (potassium) concentration changes in vitreous humor showed some success with short PMI's, however the accuracy of these results have been questioned (Coe “Vitreous potassium as a measure of the postmortem interval: an historical review and critical evaluation” Forensic Science International 1989; 42: 201-13). Other methods such measurement of force and relaxation time after direct electrical muscle stimulation with measurement of force and relaxation time were made by Madea (“Estimating time of death from measurement of the electrical excitability of skeletal muscle” Journal Forensic Science 1992;32-2:117-129)and showed consistent results in a PMI interval from 2-13 hours postmortem (hpm). McDowall et.al. (“The use of absolute refractory period in the estimation of early postmortem interval” Forensic Science International 1998; 91: 163-170) reported the use of absolute refractory period (ARP) in the estimation of the early PMI. ARP measures the ability of the nerve potentials to propagate an electrical stimulation. This is related to the loss of potential gradients in nerve cells postmortem. A linear increase in the duration of the absolute refractory period with increasing postmortem interval was observed. The value of this early marker compliments and increases the accuracy of rectal temperature methods when used in tandem in the early postmortem interval. Whener et.al. (“Delimitation of the time of death by immunohistochemical detection of thyroglobulin” Forensic Science International 2000; 110: 199-206) used thyroid tissue and did immunohistochemical staining using anti-thyroglobulin monoclonal antibodies (mAb) followed by a substrate to visualize the binding region of the mAbs. The staining is positive for the first 5 days postmortem followed by a period of mix results and after 13 days no staining was observed. Thus, a much wider PMI is addressed by this technique by using cutoffs. The authors acknowledged multiple factors that may affect denaturation of the proteins and possible decrease in antibody binding, thus, changing the cutoff values.

A more reliable time of death marker would be a great benefit in homicides, suicides, and unintentional deaths as well as other modes of death, where time of death might impact the course of a criminal investigation. Many cases involving criminal investigations revolve around the time of death and the potential alibi of a suspect. An erroneous time of death window can lead investigators down the wrong path or possibly focus a case on an innocent suspect. A more reliable time of death marker also transcends the area of criminal law and can be used in cases of disputed estates where the order of death of related family members can impact the estate's disposition.

Cardiac troponin I (cTnI), a muscle protein found in heart tissue, is the inhibitory component of the troponin ternary complex. The other two proteins in the complex are troponin C (TnC), a high affinity calcium binding protein, and troponin T (TnT), a tropomyosin binding protein. Cardiac troponin I is currently the most sensitive, useful, and reliable serum (blood) marker for the determination of damaged cardiac tissue following a heart attack or acute myocardial infarction (AMI) or other events leading to necrosis of the myocardium. The level of this protein in serum for most healthy adults is non-existent until there is a cardiac event that leads to cell death of the myocardium. Following myocardial necrosis (cardiac cell death), cTnI gets released into the bloodstream following myocardial necrosis as a result of a heart attack or myocardial infarct (MI). Nanogram levels of this protein may be monitored in serum to diagnose if the source of chest pains, whose etiology may be unknown, may be cardiac related. This test for cTnI is the “gold standard” for non-invasive MI diagnosis in the clinical diagnostics arena. Cummins et al. developed the first cTnI radioimmunoassay and showed the potential as an AMI marker in 1987 (“Cardiac specific troponin radioimmunoassay in the diagnosis of acute myocardial infarction” American Heart Journal 1987; 113:1333-44; “Cardiac specific troponin I release in canine experimental myocardial infarction: development of sensitive enzyme-linked immunoassay” Journal Molecular Cell Cardiology 1987; 19:999-1010). The first commercial immunoassay of cTnI was approved and released for medical use in the United States and Europe in 1995 and currently multiple immunoassays are available to measure the levels of this protein in serum following a suspected AMI. (Flaa et. al. “The development of a rapid, automated procedure for the determination of Troponin-I on the Stratus® Immunochemistry Analyzer” Clinical Chemistry 1993:39: 1273; Bodor et al. “Cardiac Troponin-I: A highly specific biochemical marker for myocardial infarction” Journal of Clinical Immunoassay 1994;17: 40-44). Point of care testing is now commonplace in hospital emergency departments (ED) to measure the concentration of cTnI in serum.

Cardiac troponin I is a basic protein that is also an excellent substrate for proteases such as calpains and cathepsins (Di Lisa et al. Biochemistry Journal 1995; 305: 57-61; Aoki et al. “Involvement of cathespins B and L in the post-mortem autolysis of mackerel muscle” Food Research International 1997; 30-8:585-591; Gao et al. “Role of Troponin I Proteolysis in the Pathogenesis of Stunned Myocardium” Circulation Research 1997; 80-3:393-399). Proteolysis of proteins in necrotic tissue is well documented following ischemia (Yoshida et al. Japanese Circulation Journal 1995; 59: (1) 40-48; Whipple et al. “Degradation of myofibrillar proteins by extractable lysosomal enzymes and m-calpain, and the effects of zinc chloride” Journal Animal Science 1991; 69(11): 4449-60). Troponin degradation originates in the myocardium, although, serum degradation is also present. Osuna et.al. (“Cardiac troponin I (cTnI) and the postmortem diagnosis of myocardial infarction” International Journal Legal Medicine 1998; 111(4):173-6), performed cTnI determinations in pericardial fluid of cadavers and compared it to serum levels. Both values showed statistically significant higher values for individuals who died of myocardial infarction. Morjana reported two major, stable cTnI fragments found in serum in comparison with spiked intact purified cTnI. Cardiac troponin I stability in serum is the result of a complex (TIC) formation between TnI and Troponin C (TnC) in the presence of calcium (Morjana N. “Degradation of human cardiac troponin I after myocardial infraction” Biotechnology Appl Biochem 1998; 28 (part 2):105-11; Katrukha et al. “Troponin I is released in bloodstream of patients with acute myocardial infarction not in free form but as complex” Clinical Chemistry 1997;43:8 1379-1385). Ho et.al. (“Identification of the 30 kDa polypeptide in post mortem skeletal muscle as a degradation product of troponin T” Biochimie 1994;76(5):369-375), was able to identify a Troponin T fragment in postmortem skeletal muscle as a degradation of cTnT. Huff-Lonergan et.al. (“Proteolysis of specific muscle structural proteins by mu-calpain at low pH and temperature is similar to degradation in postmortem bovine muscle” Animal Science 1996; 74(5): 993-1008), studied the proteolysis of specific of muscle structural proteins and concluded that the calpain family of proteases is responsible for postmortem degradation of muscle. It is this tissue degradation profile that is key to the use of cTnI as a time of death marker.

Cardiac troponin I differs from its skeletal isoform (sTnI) in 31 unique amino acid residues found at the N-terminus of the cardiac protein (Vallins et al. “Molecular Cloning of human cardiac troponin I using polymerase chain reaction” FEBS Letter 1990; 270:57-61; Leskzyk, et al. “Amino acid sequence of bovine cardiac troponin-I” Biochemistry 1988; 27:2821-7; Wilkinson et al. “Comparison of amino acid sequence of troponin-I from different striated muscle” Nature 1978; 271:31-5). Different groups have developed antibodies specific for cTnI exploiting these residues that are unique to the cardiac isoform (Bodor et al. “Development of monoclonal antibodies for an assay of cardiac troponin-I and preliminary results in suspected cases of myocardial infarction” Clinical Chemistry 1992; 38:2203-14; Larue et al. “New Monoclonal Antibodies as Probes for Human Cardiac Troponin I: Epitopic Analysis with Synthetic Peptides” Molecular Immunology 1992;29-2:271-278). The applicants are not aware of any literature describing the use of cTnI protein for the determination of time since death.

### SUMMARY OF THE INVENTION

The present invention is carried out by analyzing the degradation or proteolysis of a cardiac protein in the myocardium postmortem as a marker for time since death. In the postmortem interval, proteins undergo massive degradation (breakdown) via proteolytic enzymes released from muscle cells. This degradation produces different size fragments that along with intact protein may be extracted, separated, identified and analyzed using a number of different techniques. If the temporal degradation profile of these proteins is proportional to the time elapse postmortem, the time of death can be reliably predicted. The appearance of different size fragments and ratios in a PMI can increase the confidence of the prediction of time since death. This method of determination of time of death has the following advantages in forensic medicine: the heart is a well protected organ and cardiac proteins are well characterized; the technique can be automated for commercialization; the method is less temperature dependent that current state of the art, and multiple confirmation bands can increase the accuracy of estimation.

One aspect of the present invention is to create a method of determining the time of death of a cadaver for forensic pathology applications with greater accuracy. The accuracy/preciseness of time of death is improved by having multiple data points, being less temperature dependent, and having a greater range with a resolution of minutes instead of hours which is an order of magnitude better than previous methods. Specifically in this method, the proportionality of the ratio of the amount of intact protein to digested protein to the time since death provides for a more accurate estimate.

An additional aspect of the present invention is to create a time of death estimation method that is internal based and more immune to external environmental factors such as temperature fluctuations.

Another aspect of the present invention is to integrate multiple biochemical measurements, such as protein concentrations, percent degradation, temperature dependence of degradation, and the like, into the time of death measurement, to provide important supplementary information about the time of death and improve accuracy.

Yet another aspect of the present invention is to be able to automate a time of death measuring instrument, which is important both for maintaining the reliability of the measurement and for the convenience of conducting the measurement.

The above and other aspects, novel features, and advantages of the present invention will become apparent from the following detailed description of the preferred embodiments.

### BRIEF DESCRIPTION OF THE DRAWINGS

In order that the invention may be better understood, several embodiments thereof will now be described by way of example only and with reference to the accompanying drawings in which,

FIG. 1 is a western blot analysis of peak profiles of cTnI degradation extracted from bovine heart muscle.

FIG. 2 is an intensity profile of bovine cTnI intact/parent band degradation postmortem.

FIGS. 3-4 are rear views of the degradation profile of the three major band sets of postmortem bovine cTnI showing the disappearance of intact bands and appearance of fragment bands over time.

FIG. 5 illustrates the percent of bovine cTnI intact band degradation over time.

FIG. 6 is a logarithmic transformation of the percent of bovine cTnI intact band degradation measured by this method vs. time, showing the linear relationship between the two variables.

FIG. 7 illustrates how time of death can be determined independent from thermal variations in the sample. Bovine heart 1 was frozen fresh and bovine heart 2 was kept at 20° C. for 8-9 hours.

FIGS. 8-9 are western blot analyses of peak profiles of cTnI degradation extracted from human heart muscle.

FIG. 10(A) is a western blot of human cTnI postmortem profile probed with anti-cTnI monoclonal antibody. Lane identification of hours-postmortem (h pm): (1) Low molecular weight standard (2) Bovine cTnI standard (3) 0 h pm (4) 4 h pm (5) 6 h pm (5) 12 h pm (6) 24 h pm (7) 48 h pm (8) 72 h pm (9) 96 h pm (10) 120 h pm (11) 144 h pm (12) 168 h pm. FIG. 10(B) is a plot of percent human cardiac Troponin I degraded versus the log of the time postmortem. The log transformed relationship of percent cTnI degradation and the early postmortem interval (PMI) shows a pseudo-first order relationship.

FIG. 11(A) is a western blot of human standard heart: cTnI at different hours postmortem (h pm). FIG. 11(B) shows cTnI degradation profiles postmortem between human heart donors with known time since death. cTnI degradation profiles between donors show a similar degradation band pattern.

FIG. 12(A) is a western blot of postmortem human cardiac TnI degradation in heart tissue. Lane 1 represents the following hours-postmortem (h pm): Donor 1=12 h pm, Donor 2=10 h pm, Donor 3=9 h pm, Donor 4=8 h pm, Donor 5=10 h pm, Donor 6=Putrid human heart sample shows faint band for intact cTnI and mostly fragments; while Lane 2 represents an additional incubation of X+24 h pm at 20° C.+/−2° C. to observe the further cTnI degradation. FIG. 12(B) shows an Arrhenius plot of bovine cTnI degradation. FIG. 12(Inset) shows a human cTnI Arrhenius plot at 4° C.+/−2° C. and 20° C.+/−2° C. only. The graphs show a similar temperature dependence for both rates of degradation.

### DETAILED DESCRIPTION OF THE PREFERRED EMBODIMENTS

The present invention provides a new method for determining the time of death, specifically by using a cardiac protein as a time of death marker. The method of the invention is described briefly here and in more detail in the examples which follow. Generally the method of the present invention involves analyzing the degradation or proteolysis of a cardiac protein as a marker for the time since death. In the postmortem interval, proteins undergo massive degradation (breakdown) via proteolytic enzymes released from muscle cells. This degradation produces different size fragments that along with intact protein may be extracted from tissue, separated, identified, and analyzed using a variety of methods. The temporal degradation profile of this protein is proportional to the time elapse postmortem. If the ratio of percent intact protein relative to degradation fragments of the protein shows a linear relationship over time, the postmortem interval may be easily and reliably predicted. In addition, the appearance of different size fragments and ratios in a PMI can increase the confidence of the prediction of time since death. This method of determination of time of death has the following advantages in forensic medicine: the heart is a well-protected organ; many cardiac proteins are well characterized; the technique can be automated for commercialization; and it is less temperature dependent that current state of the art. This method provides a wide PMI interval with the ability to discriminate within hours of time since death.

Cadavers may be of any formerly living species, but from a utility standpoint the invention is most useful for humans. Both human and bovine hearts were used as models to establish the relationships of time of death with the concentration and fragmentation pattern of the cardiac protein.

Examples of cardiac proteins that may be monitored include, but are not limited to cTnI, cTnC, cTnT, and the like. The monitoring of cTnI in the myocardium postmortem as the cardiac protein is presently preferred because at time zero (T₀) a normal, healthy, fresh heart contains predominantly intact cTnI (i.e. a single band on a western blot).

A 1 gram heart tissue sample is easily obtained from a cadaver using a field puncture biopsy instrument. The protein may be extracted from tissue following procedures known, or readily acquired by one skilled in the art, including but not limited to homogenation of the tissue sample in a extraction buffer which prevents further degradation of the protein followed by extraction of the protein from the homogenate.

Examples of extraction buffers that may be used include, but are not limited to, 3-(N-Morpholino)propane sulfonic acid (MOPS), N-Tris-hydroxymethyl methyl-2-aminoethane sulfonic acid (TES), 3-[N-bis(hydroxymethyl) amino]-2-hydroxypropane sulfonic acid (DIPSO), Piperazine-N, N′Bis(2)hydroxypropane sulfonic acid (HEPPSO), Tris-(hydroxymethyl)aminoethane, N-2-Hydroxyethylpiperazine-N′-2-aminoethane sulfonic acid (HEPES), 3-[N-Tris-hydroxymethyl)methylamino]-2-hydroxypropane sulfonic acid (TAPSO), (2p[2-Amino-2-oxoethyl)-aminoethanesulfonic acid (ACES), Tris, sodium phosphate, and the like. A presently preferred buffer is Tris, sodium phosphate. The concentration of the buffer solution may range from about 5 mM to about 200 mM. It is presently preferable that the buffer concentration is about 90 to about 110 mM.

Preferably the homogenate should include a cocktail of protease inhibitor(s) to prevent further degradation of the marker protein. Examples of commercially available protease inhibitor cocktails include, but are not limited to, Sigma Protease Inhibitor Cocktail, Aprotinin, EDTA, pefabloc, [(S)-1-Carboxy-2-Phenyl]-carbamoyl-Arg-Val-arginal (antipain-HCL), Phe-(Cap)-Leu-Phe-al-[(S)-1-carboxy-isopentyl)-carbamoyl-alpha-(2-iminohexahydro-4(S)-pyrimidyl]-L-glycyl-L-phenylalaninal (chymostatin), N-[N-(L-trans-carboxyoxiran-2-carbonyl)-L-leucyl]-agmatine(E-64),leupeptin,[(2S,2R)-3-Amino-2-hydroxy-4-Phenylbutanoyl]-L-Leucine(pepstatin), Phenylmethylsulfonyl fluoride (PMSF), Di-isopropylfluorophosphate (DFP), 4-Amidino-Phenyl)-Methane-Sulfonyl Fluoride (APMSF), Tosyl Lysyl ChloromethylKetone (TLCK), 1-Chloro-3-tosylamido-4-phenyl-2-butanone, Boehringer Mannheim Complete Protease Inhibitor, and the like. A presently preferred protease inhibitor cocktail is Boehringer Mannheim's Complete Protease Inhibitor. The concentration of these protease inhibitor cocktails in buffer range from about 0.5 to 200 mM and it is presently preferable that the protease inhibitor cocktail concentration range from about 1 to about 50 mM.

When cTnI is the marker protein it is presently preferable to include an additional salt to the extraction buffer salts to help solubilize the protein. Examples of additional salts include, but are not limited to potassium chloride, calcium chloride, lithium chloride, sodium chloride, and the like. The use of sodium chloride as the additional salt is presently preferred. Additional salt concentrations can range from about 5 to about 400 mM and it is presently preferred that the additional salt concentration is about 190 to about 210 mM.

Optionally a preservative may be also used as a component of the extraction buffer solution. Examples of preservatives include, but are not limited to, clotrimazole, chlroamphenicol, gentamicin, mycostatin, thimerasol, sodium azide, Kathon, and the like. A presently preferred preservative is sodium azide. Concentrations of preservative may range from about 0.01% to 0.2% and it is presently preferred that the preservative concentration range from about 0.05 to about 0.15%.

The protein may be extracted from the homogenate following procedures known, or readily acquired by one skilled in the art, including but not limited to the use of capture microparticles. A presently preferred method is using magnetic microparticles with carboxylate functionalities for charge capture of the protein. In addition, capture microparticles may be prepared containing antibodies which are specific for the marker protein. These microparticles may be prepared following procedures known, or readily acquired by one skilled in the art including, but not limited to, covalently linking an amine moiety of the monoclonal antibody through (1-ethyl-3-[3-dimethylaminopropyl]carboiimide hydrochloride and N-hydroxy succinimide (EDC/NHS) activation of carboxyl modified beads, passive adsorption of the antibody onto the surface of the bead, and the like.

The bead or conjugate may then be washed with an extraction buffer to release the marker protein. Examples of elution buffers include, but are not limited to, 3-(N-Morpholino)propane sulfonic acid (MOPS), N-Tris-hydroxymethyl methyl-2-aminoethane sulfonic acid (TES), 3-[N-bis(hydroxymethyl) amino]-2-hydroxypropane sulfonic acid (DIPSO), Piperazine-N, N′Bis(2)hydroxypropane sulfonic acid (HEPPSO), Tris-(hydroxymethyl)aminoethane, N-2-Hydroxyethylpiperazine-N′-2-aminoethane sulfonic acid (HEPES), 3-[N-Tris-hydroxymethyl)methylamino]-2-hydroxypropane sulfonic acid (TAPSO), (2p[2-Amino-2-oxoethyl)-aminoethanesulfonic acid (ACES), Tris, sodium phosphate, sodium acetate, formic acid, acetic acid, lithium acetate, and amino acids, such as any of the natural amino acids, and the like. A presently preferred amino acid-based elution buffer is glycine. Elution buffer concentrations may range from about 10 to about 400 mM. A presently preferred elution buffer concentration is about 100 mM. The pH of the elution buffer may range from about 0 to about 6. A presently preferred pH is about 1 to about 3. Typically, from about 1 to about 10 washes of extraction buffer are done and it is presently preferred to do about 2 to about 5 washes with extraction buffer.

When cTnI is the marker protein it is presently preferable to include an additional salt to the extraction buffer salts to help solubilize the protein. Examples of additional salts include, but are not limited to potassium chloride, calcium chloride, lithium chloride, sodium chloride, and the like. The use of sodium chloride as the additional salt is presently preferred. Additional salt concentrations can range from about 5 to about 800 mM and it is presently preferred that the additional salt concentration is about 150 to about 250 mM.

It is presently preferable to include a chaotrope or denaturant in the elution buffer. Examples of chaotropes or denaturants include, but are not limited to, guanidine hydrochloride and sodium dodecyl sulfate (SDS), urea, and the like. A presently preferred chaotrope is urea. Chaotrope concentration may be in the range from about 1 to about 8M. A presently preferred chaotrope concentration is about 5 to about 7M.

The protein and its fragments may then be further separated following a variety of chromatographic techniques well known in the art. Examples of separation techniques that may be used include, but are not limited to, isoelectric focusing, native gel electrophoresis, HPLC size exclusion, FPLC size exclusion, capillary electrophoresis, LC/MS-ESI, MALDI/TOF/MS, CM-Sepharose cation exchange chromatography for HPLC or FPLC, and the like. A presently preferred method is using 15% SDS-PAGE (reducing conditions) with a concentration of gel from about 7 to about 20% PAGE followed by transfer to (PVDF) paper by western blot.

The separated proteins may then be probed and identified by a variety of methods well known in the art including, but not limited to, microarray/chip methods, automated partitioning enzyme immunoassay methods, affinity antibody capillary electrophoresis and flow cytometry techniques with multiple dyes, and the like. Examples of probes that may be used include, but are not limited to, monoclonal or polyclonal antibodies, whole IgG or F(ab)′ (digested), dual antibody conjugate-ALP or single antibody directly conjugated to enzyme with heterobifunctional cross-linker with or without a label, and the like. Examples of labels that may be used to visualize and identify the proteins include, but are not limited to label enzyme systems such as ALP (alkaline phosphatase), HRP (horseradish peroxidase), B-Gal (galactosidase) or radioactive unstable isotopes label (iodine or phosphorus), and the like. The labels may be detected by a variety of methods well known in the art including calorimetric, fluorogenic, chemilluminescent, gamma-counter (radioactive), laser-induced fluorescence, precipitating substrates (NBT/BCIP), using Coommassie brilliant blue R-250 stain, and the like. A presently preferred method is incubating a western blot with cardiac specific monoclonal antibodies followed by an anti-mouse antibody labeled with alkaline phosphatase (anti-mAb-ALP). This conjugate is washed off a BCIP/NBT is developed until the bands are visible.

The data may then analyzed by a variety of techniques well known in the art including but not limited to, comparing the ratio of fragment peptides to intact protein, the ratio of fragments to fragments, the ratio of fragments to a standard, the concentration of fragments to a reference internal standard, fragmentation patterns, using certain bands as cutoff standards for certain days that these bands appear, and the like. A presently preferred method is calculating the percent degradation of the protein vs. the log of hours post mortem.

The following examples are provided to further illustrate specific aspects and practices of this invention. These examples describe particular embodiments of the invention, but are not to be construed as limitations on the scope of the present invention or the appended claims.

Fresh bovine hearts were obtained from Pel-Freez Biologicals, Rogers, AK. Antibodies used to probe western blots were a gift from Dade Behring, Inc., Miami, Fla. The human tissue samples were obtained from the National Disease Research Interchange (NDRI), Philadelphia, Pa. A heart tissue sample was a gift from the University of Miami School of Medicine-Transplant Unit, Miami, Fla. The magnetic microparticles (CM-MP cat#24152105050250) were purchased from Seradyn a division of Alexon-Trend, Indianapolis, Ind. An FB-VE16-1 (16×14 cm) electrophoresis system, Fisher Biotech and PVDF Western blot paper were purchased from Pittsburgh, Pa. SDS-PAGE Prestained broad range molecular weight markers (cat#161-0318), blocking casein solutions, anti-mouse IgG-ALP conjugate (cat#170-6520), BCIP/NBT substrate for blots (cat#170-6532/39) were from Bio-Rad Laboratories, Hercules, Calif. Tissue Tearer. All chemicals unless otherwise specified were obtained from Sigma-Aldrich, St. Louis, Mo. Human Heart tissue was stored at −80 C and bovine hearts were stored at −20 C until analysis.

### EXAMPLE 1

A 1 g cardiac tissue sample is taken from the corpse of a bovine. The cardiac tissue can be frozen for later analysis or homogenized with 4 mL of extraction buffer (10 mM sodium phosphate, 100 mM Tris, 200 mM sodium chloride, 0.1% sodium azide, 1 tablet of Boehringer Mannheim Complete® Protease Cocktail Inhibitor in 50 mL, pH=8.0). The samples are then centrifuged at 5000 g for 5 minutes. The supernatant is decanted and mixed with 50 uL of 5% (w/w) magnetic microparticles and incubated on a rotating wheel for 1 hour. These microparticles are coupled to antibodies (anti-cTnI antibodies) and are used for extraction of the cTnI from the complex homogenate. The microparticles are added to the homogenate and after an incubation period they are removed. The microparticles are washed and the protein is eluted with elution buffer Y. The beads are then centrifuged and washed with extraction buffer 3 times. Bound cTnI is then eluted off the beads with elution buffer (100 mM glycine, 6M urea, 250 mM sodium chloride, pH=2 ). The eluted protein is diluted (1:1) with 5×SDS-PAGE sample buffer (2% SDS, 0.0625M Tris-HCL (pH 6.8), 5% 2-B-mercaptoethanol, 10% glycerol, 0.002% bromophenol blue) and pH adjusted with 6M sodium hydroxide. Samples are then boiled at 100° C. for 3 minutes. 15% SDS-PAGE electrophoresis was run for 500 Vh at 140V for 3.5 (hours as described by Laemmli (“Cleavage of structural proteins during the assembly of the head of bacteriophage T4” Nature 1970; 227:680-685). The completed gel is then transferred to polyvinylidene difluoride (PVDF) paper using a semi-dry Western blot technique. The buffers for the western blot are as follows: Anode buffer 1: 300 mM Tris, 20% methanol, pH 10.4. Anode buffer 2: 25 mM Tris, 20% methanol, pH 10.4; Cathode buffer: 25 mM Tris, 20% methanol, 40 mM 6-aminohexanoic acid, pH not adjusted. The blot is run for 45 min at 240 mA. After running the western blot, the membrane is blocked by adding blotting grade casein protein for 4 hours, then the membrane is probed with anti-cTnI mouse monoclonal antibody (2 ug/mL) for 4 hours followed by the anti-mouse-alkaline phosphatase (ALP) conjugate (1:500). The blot is developed with using BCIP/NBT alkaline phosphatase substrate. The blot is then dried, and the cTnI bands and its fragments are scanned, analyzed, and quantified using Sigma Scan® software.

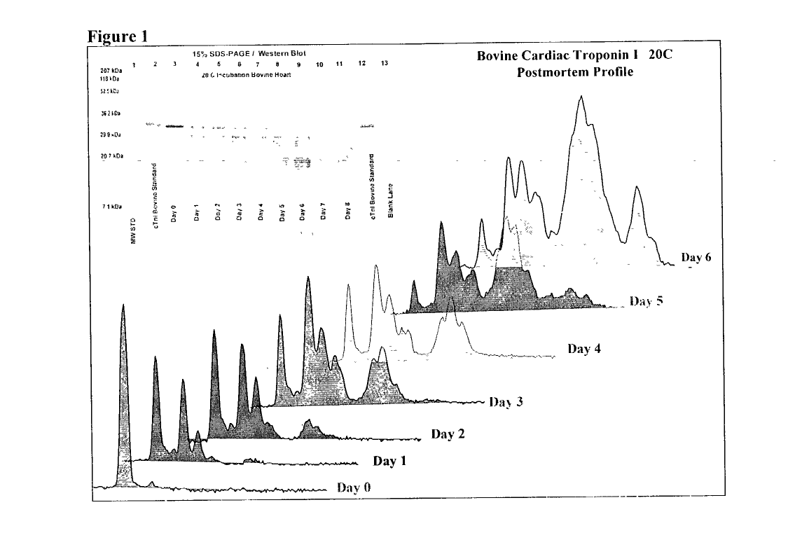

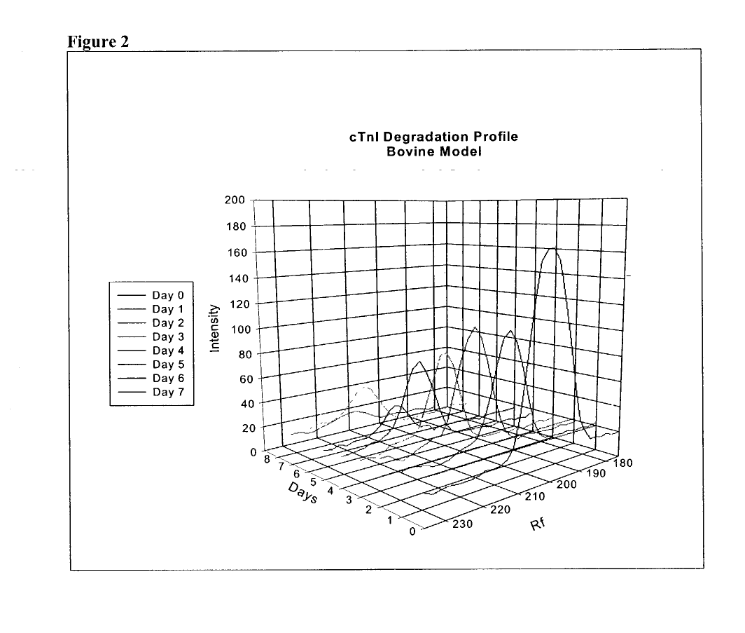

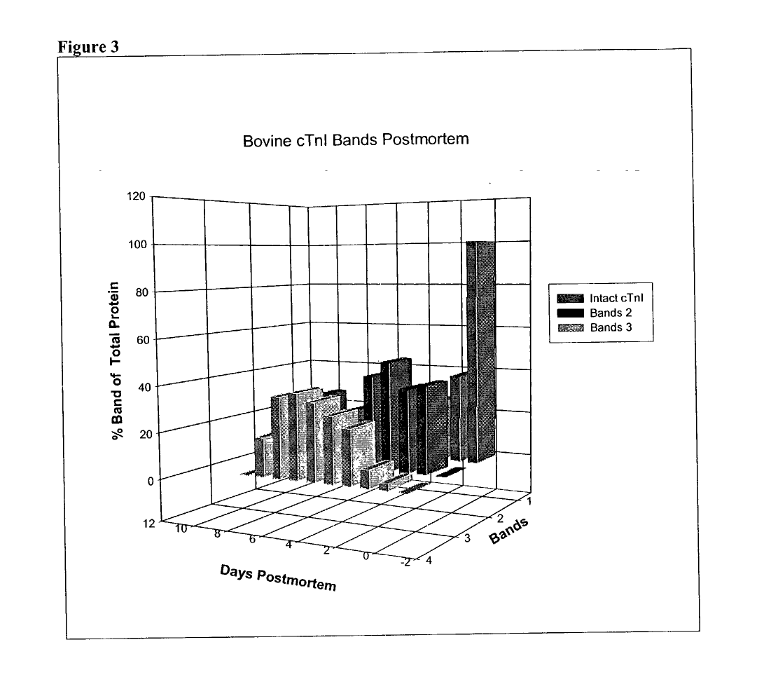

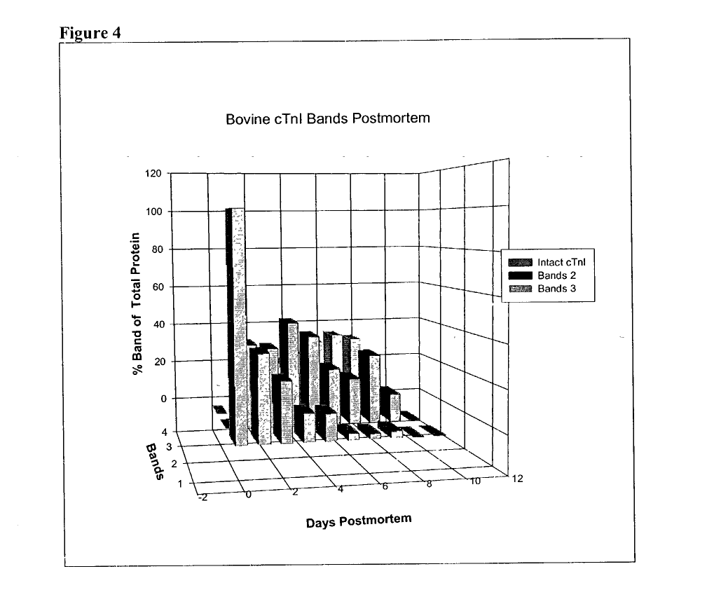

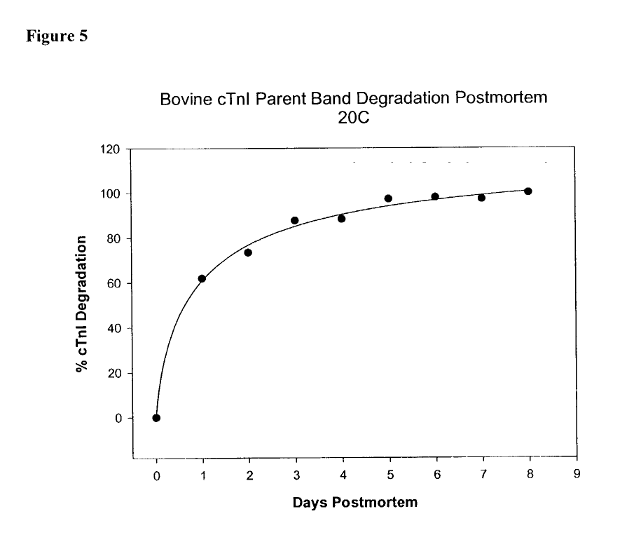

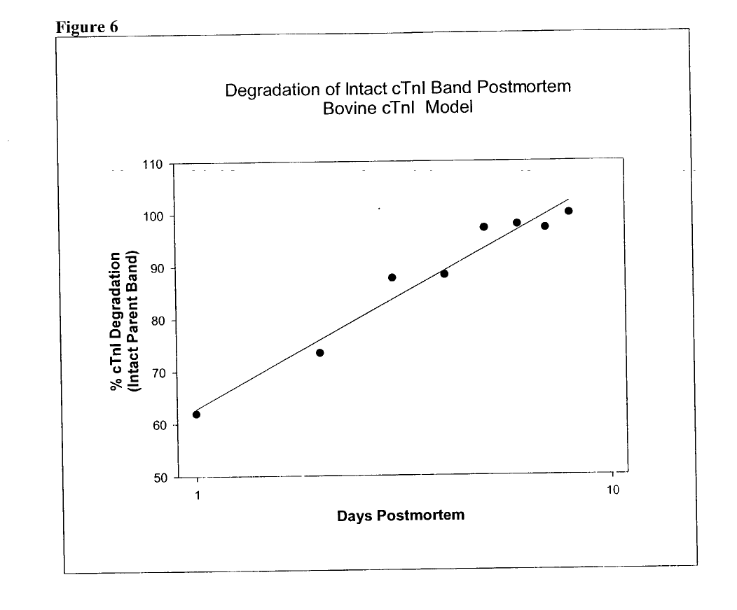

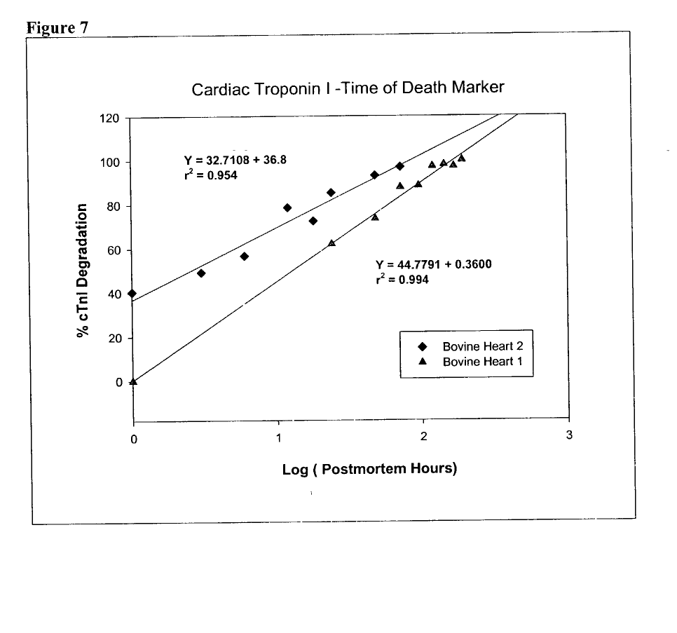

FIG. 1 shows a typical profile of heart tissue incubated at room temperature equivalent to a post mortem interval of about 0 to 8 days. FIG. 2 shows the disappearance of intact cardiac troponin I post mortem. The antibody epitopes (binding region) is typically well preserved through the degradation if they are cardiac specific antibodies. There are numerous companies that sell cardiac troponin I specific monoclonal antibodies. FIG. 3 shows the disappearance of the intact cTnI band and the emergence of other smaller molecular weight polypeptide fragments bands as time postmortem increases. FIG. 4 shows the relationship between the percent degradation of cTnI and time postmortem. This relationship was then transformed by taking the percent cTnI degradation versus the log of the hours postmortem. This gives a linear relationship that has a strong correlation between time of death and degradation of cTnI. FIG. 6 shows the actual power of the technique by taking heart #2 at Time=0 (T₀) and reading it off the standard curve of the fresh heart #1. The data reveals that heart #2 was at 20 C for about 8-10 hours. The extra points for heart #2 were performed to increase the accuracy of the y-intercept. In reality only one sample is needed to determine the time postmortem, T₀. T₀ is the time when the cadaver heart tissue is sampled and frozen for later analysis. The confidence of the prediction may also be increased by using certain bands as cutoffs, for days that these bands appear.

### EXAMPLE 2

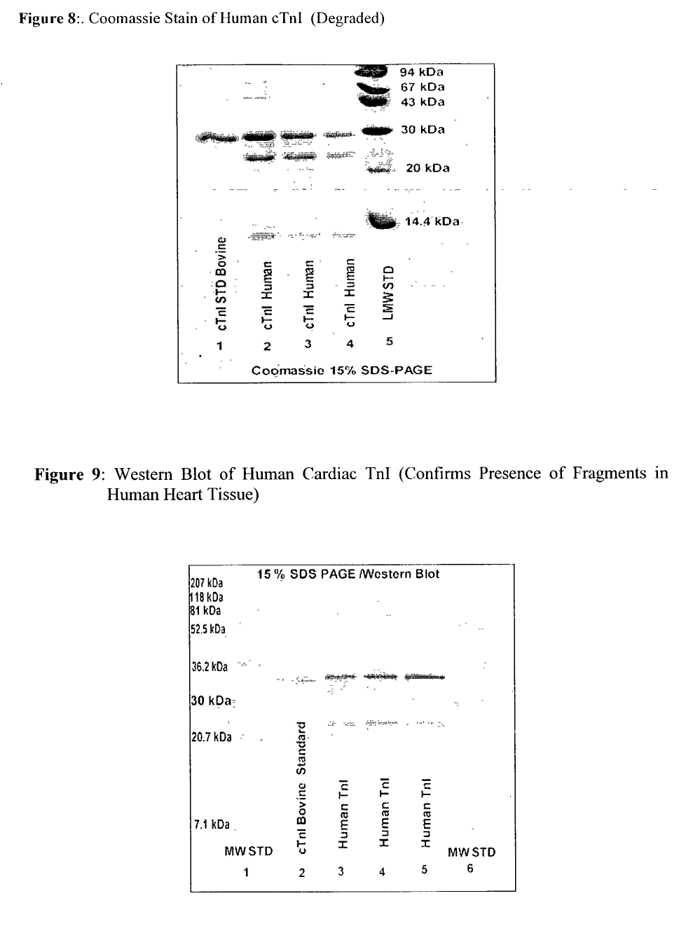

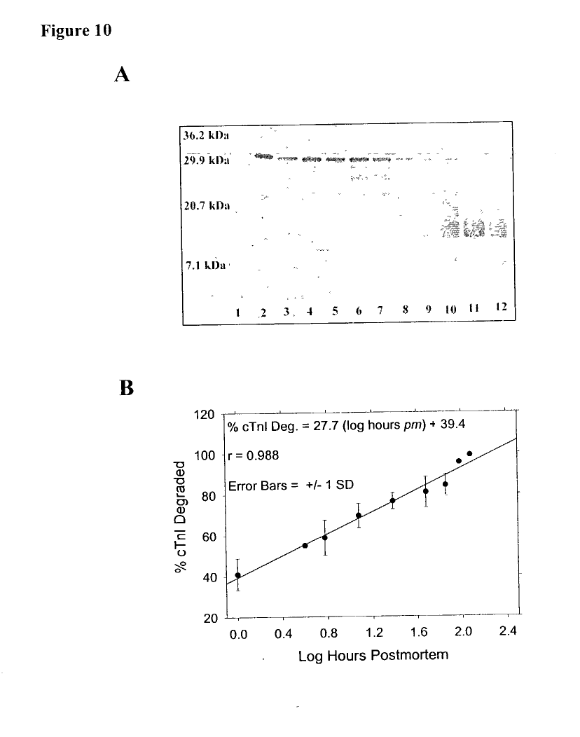

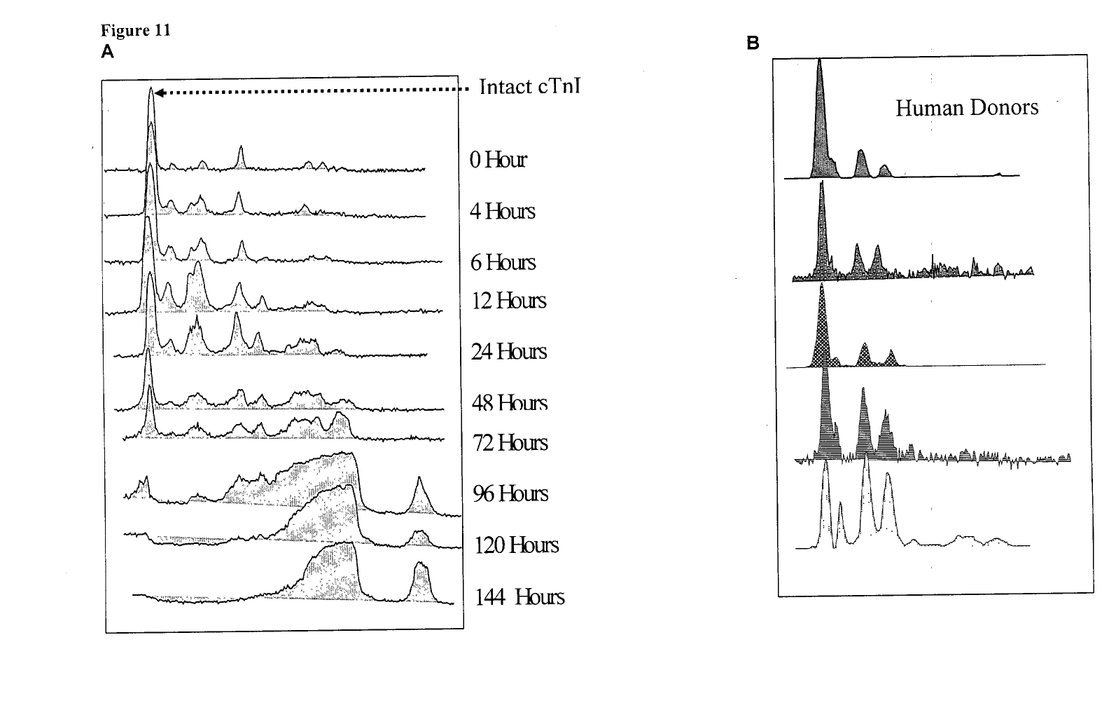

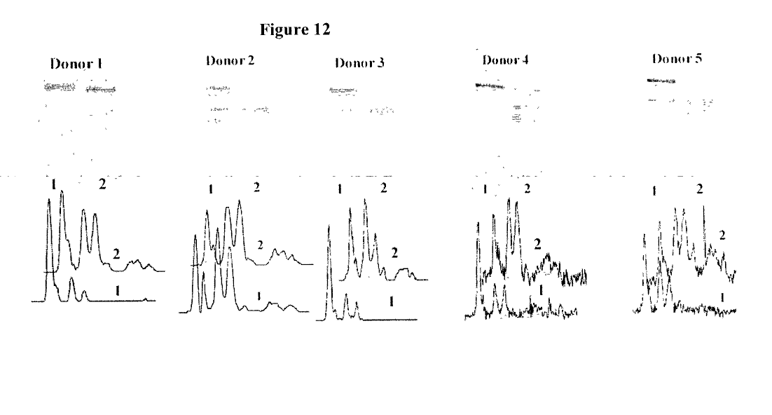

A similar profile is seen for human cTnI as shown in FIGS. 8-12 and Table 1. FIG. 8 shows a human cardiac Troponin I protein that was purposely degraded to obtain some fragmentation. The stain used was Coomassie Brilliant Blue, which is not protein specific. FIG. 9 shows the same gel that was transferred by Western blot and probed with cardiac specific antibodies used in the bovine model mentioned above. FIG. 10 shows the pseudo-first order relationship of the log transformed data of percent cTnI degradation and the early postmortem interval (PMI). FIG. 11 shows the similar degradation band pattern of cTnI from human heart donors with known times of death. FIG. 12 compares the temperature dependence for both human and bovine cTnI rates of degradation. Table 1 compares the qualitative and semi-quantitative approaches for estimation of time of death with cardiac troponin I. Donor 6 in Table 1 was a putrid smelling sample that showed a time different than the stated time postmortem. That appears to be a compromised sample that the test properly identified. This data is consistent with the bovine (cow) heart data above. This shows that the use of Cardiac Troponin I as a time of death marker works just as well for human cardiac tissue.

TABLE 1
Comparison of Qualitative versus Semi-quantitative Approach for Estimation of
Time of Death with Cardiac Troponin I.

| Tissue | Time | Standard Human Heart Postmortem Intervals | | | | | | | | |
|---|---|---|---|---|---|---|---|---|---|---|
| Human | Postmortem | Qualitative (hours) at 20+/−2C | | | | | | | | |
| Donors | (Hours) | 0 to 4 | 4 to 6 | 6 to 12 | 12 to 24 | 24 to 48 | 48 to 72 | 72 to 96 | 96 to 120 | 120 to 144 |
| 1 | 12 | | | x(5) | | | | | | |
| | 36 | | | | | x(36) | | | | |
| 2 | 10 | | | | x(36) | | | | | |
| | 34 | | | | | x(51) | | | | |
| 3 | 9 | | x(5) | | | | | | | |
| | 33 | | | | | x(33) | | | | |
| 4 | 8 | | | x(5) | | | | | | |
| | 32 | | | | | x(33) | | | | |
| 5 | 10 | | | | x(10) | | | | | |
| | 34 | | | | | x(47) | | | | |
| 6 | 8 | | | | | | | x(105) | | |
| | 32 | | | | | | | | | x(>105) |

X = Qualitative results
( ) = Semiquantitative results (hours)

The results obtained to date show that there is a significant potential for this protein to be a time of death marker. It can be used for hourly and daily monitoring of the short postmortem interval (0 to 200 hours). The extraction protocol minimizes proteolysis of the protein in the handling and isolation of the protein. These techniques can be also automated for commercialization using a diverse number of analytical techniques.

The above examples demonstrate the novelty and utility of the method of determining time of death of the present invention. Every reference cited herein is hereby incorporated by reference in its entirety. The foregoing detailed description of the preferred embodiments of the invention has been given for clearness of understanding only, and no unnecessary limitations should be understood therefrom, as modifications will be obvious to those skilled in the art. Variations of the invention as hereinbefore set forth can be made without departing from the scope thereof, and, therefore, only such limitations should be imposed as are indicated by the appended claims.

What is claimed is:

1. A method of determining the time of death of a corpse comprising the step of comparing known values for amounts of fragmented or intact cardiac protein found in a mammal with time since death of said mammal with the amount of fragmented or intact cardiac protein to fragmented cardiac protein taken from said corpse at a given time.

2. A method of determining the time of death of corpse in accordance with claim 1 wherein the protein is cTnI.

3. A method of determining the time of death in accordance with claim 2, wherein said organism is a human.

4. A method of determining the time of death of a corpse in accordance with claim 1 further comprising the steps of:
(a) taking a cardiac tissue sample from a corpse;
(b) homogenizing the cardiac sample in a first buffer solution;
(c) extracting a cardiac protein from the homogenate;
(d) separating the proteins;
(e) transferring the separated proteins to a device for detection;
(f) detecting an amount of cardiac protein selected from the group consisting of fragmented cardiac protein, intact cardiac protein and a ratio of intact cardiac protein to fragmented cardiac protein;
(g) analyzing the value obtained in step f with known values for amounts of fragmented or intact cardiac protein found in a mammal per unit time after death, to determine the time of death of said corpse.

5. A method of determining the time of death in accordance with claim 4, wherein said extracting step is accomplished by incubation with magnetic microparticle selected from the group consisting of a microparticle covalently bound to anti-cardiac protein antibodies, a magnetic microparticle containing charged groups, and a magnetic microparticle adsorbed with antibodies.

6. A method of determining the time of death in accordance with claim 4, wherein said extracting step is accomplished by microparticulate-bound anti-cTnI antibodies.

7. A method of determining the time of death in accordance with claim 4, wherein said separating step is accomplished with SDS-PAGE electrophoresis.

8. A method of determining the time of death in accordance with claim 4, wherein said transferring step is accomplished by western blotting onto PVDF paper.

9. A method of determining the time of death in accordance with claim 4, wherein said detecting, visualizing and quantifying step is accomplished with anti-cTnI antibodies conjugated to a marker enzyme.

10. A method of determining the time of death in accordance with claim 9, wherein said marker enzyme is alkaline phosphatase.

11. A device which automates the method in accordance with claim 4 comprising:
(a) a homogenation unit which allows introduction and removal of a sample;
(b) a means extracting a cardiac protein from the homogenate;
(d) a means of separating the proteins;
(e) a means of transferring the separated proteins to a device for detection;
(f) a means of detecting, visualizing, and quantifying the amount of protein;
(g) a means of comparing and analyzing the values obtained with standard protein concentration and fragmentation patterns per unit time after death.

12. The use of a device in accordance with claims 11 in biological, biochemical, or chemical testing.

13. A method of determining time of death of a corpse in need thereof using a device in accordance with claim 11 comprising the steps of:
(a) determining standard values for a cardiac protein's fragmentation per unit time after death;
(b) determining the fragmentation pattern for said cardiac protein from said corpse;
(c) determining the time of death of said corpse by comparing the values of step b with the values of step a.
